(19) United States
(12) Patent Application Publication
(10) Pub. No.: US 2025/0375414 A1
(43) Pub. Date: Dec. 11, 2025

(54) UROLITHIN COMBINATIONS

(71) Applicant: Amazentis SA, Lausanne (CH)

(72) Inventors: Anurag Singh, Lausanne (CH); Julie Faitg, Lausanne (CH); Davide D'Amico, Renens (CH); Christopher L. Rinsch, Lausanne (CH)

(21) Appl. No.: 19/235,287

(22) Filed: Jun. 11, 2025

(30) Foreign Application Priority Data
Jun. 11, 2024 (GB) 2408320.6

(51) Int. Cl. A61K 31/37; A61K 31/00; A61K 31/047; A61K 31/198; A61K 31/352; A61K 31/4045; A61K 31/4172; A61K 31/4415; A61K 31/519; A61K 31/685; A61K 31/714; A61K 33/04; A61K 33/30; A61K 36/185; A61K 36/324; A61K 36/41; A61K 36/81; A61P 25/28; A61P 29/00; A61P 37/02

(52) U.S. Cl. CPC A61K 31/37; A61K 31/047; A61K 31/198; A61K 31/352; A61K 31/4045; A61K 31/4172; A61K 31/4415; A61K 31/519; A61K 31/658; A61K 31/685; A61K 31/714; A61K 33/04; A61K 33/30; A61K 36/185; A61K 36/324; A61K 36/41; A61K 36/81; A61P 25/28; A61P 29/00; A61P 37/02

(57) ABSTRACT

The invention relates to compositions comprising urolithins with active agents, particularly compositions comprising urolithins and agents which promote brain health and/or immune health. The invention also relates to such compositions, for use for the treatment or prevention of diseases, disorders and conditions, and for use in non-therapeutic treatments. Example of agents which promote brain health include brain protective agents (for example phosphatidylserine, phosphatidylcholine and/or choline), agents that enhance neurotransmission (for example a nootropic, for example ergothioneine, huperzine, aGPC, taurine and/or L-theanine), agents that promote neurogenesis (for example selenium); and brain essential nutrients (example Vitamin B6, B9, B12, magnesium salts, folic acid and/or L-methylfolate). Examples of agent which promote immune health include quercetin, Elderberry or an extract thereof; a selenium salt; and/or a zinc salt.

### RELATED APPLICATION

This application claims the benefit of priority to UK Patent Application No. 2408320.6, filed Jun. 11, 2024.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, al-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

### SUMMARY

The invention relates to compositions comprising urolithins with active agents, particularly compositions comprising urolithins and agents which promote brain health and/or immune health. The invention also relates to such compositions, for use for the treatment or prevention of diseases, disorders and conditions, and for use in non-therapeutic treatments.

As discussed above, urolithins, for example, urolithin A, are effective in a variety of conditions, and for improving health, in individuals of all ages, particularly for promoting healthy aging and increasing healthspan. However, there is a continuing need for improvement and we have identified a number of combinations of urolithins with other active ingredients, which can surprisingly increase the benefit of urolithin administration.

Therefore, according to the first embodiment of the invention there is provided a composition comprising:
(a) a compound of formula (I), or a salt, prodrug, or pharmaceutically acceptable salt thereof;

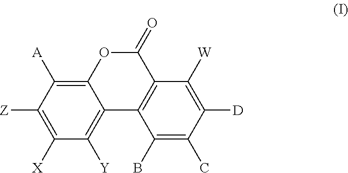

wherein:
   A, B, C, D, W, X, Y and Z are each independently selected from H and OH; and
(b) one of more agents selected from agents for improving brain health and/or agents for improving immune health.

In a further embodiment of the invention, there is provides a composition comprising:
(a) a compound of formula (I), or a salt, prodrug, or pharmaceutically acceptable salt thereof; and
(b) one of more agents selected from agents for improving brain health.

In a further embodiment of the invention, there is provides a composition comprising:
(a) a compound of formula (I), or a salt, prodrug, or pharmaceutically acceptable salt thereof; and
(b) one of more agents selected from agents for improving immune health.

The compositions of the invention are surprisingly effective and efficient for the provision of benefits to a subject taking the composition. The invention is described below in further detail.

### BRIEF DESCRIPTION OF THE FIGURES

FIG. 1: Interleukin-6 (IL-6) concentration, expressed as (pg/ml) measured in culture media from C2C12 myotubes treated for 24 hours with Urolithin A (UA) at the indicated doses and then treated with cytomix (CTX) for further 24 hours. Data are expressed as mean+/−SEM. One-way ANOVA.

FIG. 2: Interleukin-6 (IL-6) concentration, expressed as (pg/ml) measured in culture media from C2C12 myotubes treated for 24 hours with UA and ERGO at the indicated doses and then treated with cytomix (CTX) for further 24 hours. Data are expressed as mean+/−SEM. One-way ANOVA.

FIG. 3: Interleukin-6 (IL-6) concentration, expressed as (pg/ml) measured in culture media from C2C12 myotubes treated for 24 hours with UA and ERGO at the indicated doses and then treated with cytomix (CTX) for further 24 hours. Data are expressed as mean+/−SEM. One-way ANOVA.

FIG. 4: Interleukin-6 (IL-6) concentration, expressed as (pg/ml) measured in culture media from C2C12 myotubes treated for 24 hours with UA and ERGO at the indicated doses and then treated with cytomix (CTX) for further 24 hours. Data are expressed as mean+/−SEM. One-way ANOVA.

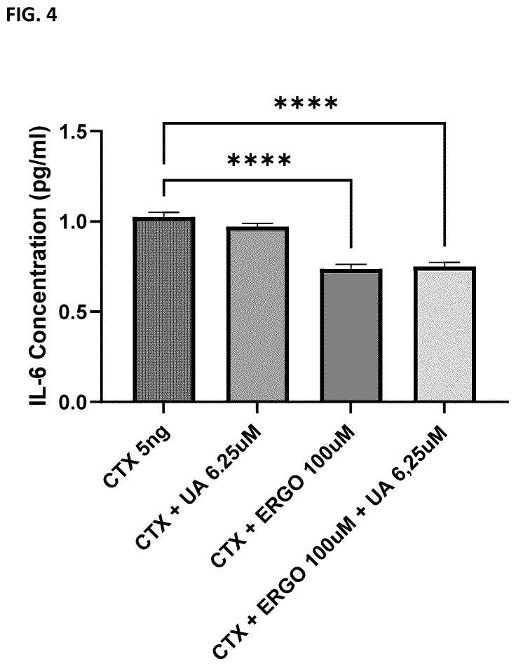

### DETAILED DESCRIPTION

### Compositions Comprising an Agent for Improving Brain Health

In an embodiment of the invention, there is provides a composition comprising:
(a) a compound of formula (I), or a salt, prodrug, or pharmaceutically acceptable salt thereof; and
(b) one of more agents selected from an agent for improving brain health.

An agent for improving brain health may be selected from:
(i) brain protective agents (for example phosphatidlyserine, phosphatidylcholine or choline);
(ii) agents that enhance neurotransmission (for example a nootropic, for example ergothioneine, huperzine, aGPC, taurine or L-theanine);
(iii) agents that promote neurogenesis (for example selenium); and/or
(iv) brain essential nutrients (example Vitamin B6, B9, B12, magnesium salts, folic acid or L-methylfolate)

The composition may also comprise a further mitophagy activator and/or a mitochondrial function boosting agent (for example an NAD booster, resveratrol, CoQ10 or pyrroloquinoline quinone (PQQ))

In an embodiment of the invention, there is provided a composition comprising:
(a) a compound of formula (I), or a salt, prodrug, or pharmaceutically acceptable salt thereof; and
(b) a brain protective agent.

The brain protective agent may be an agent supporting brain function, for example, an agent which maintains or reinforces myelin sheaths. Such an agent may be selected from phosphatidylserine, phosphatidylcholine, choline, lecithin, Eicosapentanoic acid (EPA), Docosahexanoic acid (DHA) and/or a phospholipid complex. A suitable phospholipid complex may be a complex comprising, phosphatidylcholine, choline and/or phytosomes together with a magnesium salt. In particular, the brain protective agent is phosphatidylserine.

In an embodiment of the invention, there is provided a composition comprising:
(a) a compound of formula (I), or a salt, prodrug, or pharmaceutically acceptable salt thereof; and
(b) phosphatidylserine.

Sources of phosphatidylserine are commonly from plant sources. For example, from soy lecithin, sunflower lecithin or cabbage lecithin. In one embodiment, the phoshatidylserine, is NeuroPeak (Frau Pharma srl, Milan, Italy); phosphatidylserine from soy or sunflower.

In an embodiment of the invention, there is provided a composition comprising:
(a) a compound of formula (I), or a salt, prodrug, or pharmaceutically acceptable salt thereof; and
(b) an agent that enhances neurotransmission.

The agent that enhances neurotransmission may be a nootropic or a cognitive enhancer. Such an agent may be selected from ergothioneine, huperzine (for example huperzine A), L-alpha-glycerylphosphorylcholine (Alpha-GPC), taurine or L-theanine. In particular, the agent that enhances neurotransmission is ergothioneine.

In an embodiment of the invention, there is provided a composition comprising:
(a) a compound of formula (I), or a salt, prodrug, or pharmaceutically acceptable salt thereof; and
(b) ergothioneine.

In an embodiment of the invention, there is provided a composition comprising:
(a) a compound of formula (I), or a salt, prodrug, or pharmaceutically acceptable salt thereof; and
(b) an agent that promotes neurogenesis.

The agent that promotes neurogenesis may be selected from selenium, melatonin or chromium. In particular, the agent that promotes neurogenesis is selenium.

In an embodiment of the invention, there is provided a composition comprising:
(a) a compound of formula (I), or a salt, prodrug, or pharmaceutically acceptable salt thereof; and
(b) selenium.

In an embodiment of the invention, there is provided a composition comprising:
(a) a compound of formula (I), or a salt, prodrug, or pharmaceutically acceptable salt thereof;
(b) a brain protective agent; and
(c) an agent that enhances neurotransmission.

The brain protective agent may be selected from the brain protective agents that are disclosed above. The agent that enhances neurotransmission may be selected from the agents that enhance neurotransmission that are disclosed above.

In particular, there is provided a composition comprising:
(a) a compound of formula (I), or a salt, prodrug, or pharmaceutically acceptable salt thereof;
(b) phosphatidylserine; and
(c) ergothioneine.

In an embodiment of the invention, there is provided a composition comprising:
(a) a compound of formula (I), or a salt, prodrug, or pharmaceutically acceptable salt thereof;
(b) a brain protective agent; and
(c) an agent that promote neurogenesis.

The brain protective agent may be selected from the brain protective agents that are disclosed above. The agent that promotes neurogenesis may be selected from the agents that agents that promote neurogenesis that are disclosed above.

In particular, the invention provides a composition comprising:
(a) a compound of formula (I), or a salt, prodrug, or pharmaceutically acceptable salt thereof;
(b) phosphatidylserine; and
(c) selenium.

In an embodiment of the invention, there is provided a composition comprising:
(a) a compound of formula (I), or a salt, prodrug, or pharmaceutically acceptable salt thereof;
(b) an agent that enhances neurotransmission; and
(c) an agent that promote neurogenesis.

The agent that enhances neurotransmission may be selected from the agents that enhance neurotransmission that are disclosed above. The agent that promotes neurogenesis may be selected from the agents that promote neurogenesis that are disclosed above.

In particular, there is provided a composition comprising:
(a) a compound of formula (I), or a salt, prodrug, or pharmaceutically acceptable salt thereof;
(b) ergothioneine; and
(c) selenium.

In an embodiment of the invention, there is provided a composition comprising:
(a) a compound of formula (I), or a salt, prodrug, or pharmaceutically acceptable salt thereof;
(b) a brain protective agent;
(c) an agent that enhances neurotransmission; and
(d) an agent that promotes neurogenesis.

In particular, there is provided a composition comprising:
(a) a compound of formula (I), or a salt, prodrug, or pharmaceutically acceptable salt thereof;
(b) phosphatidylserine;
(c) ergothioneine; and
(d) selenium.

In a further embodiment, there is provided a composition comprising:
1) about 100 mg to about 1000 mg urolithin A;
2) about 50 mg to about 500 mg phosphatlidylserine;
3) about 1 mg to about 100 mg ergothioneine; and
4) about 1 μg to about 250 μg selenium.

In a further embodiment, there is provided a composition comprising:
1) about 100 mg to about 500 mg urolithin A;
2) about 100 mg to about 250 mg phosphatlidylserine;
3) about 5 mg to about 50 mg ergothioneine; and
4) about 10 μg to about 100 μg selenium.

In a further embodiment, there is provided a composition comprising:
1) about 100 mg to about 500 mg urolithin A;
2) about 100 mg to about 200 mg phosphatlidylserine;
3) about 5 mg to about 30 mg ergothioneine; and
4) about 10 μg to about 50 μg selenium.

In a further embodiment, there is provided a composition comprising:
1) about 150 mg to about 340 mg urolithin A;
2) about 150 mg to about 195 mg phosphatlidylserine;
3) about 12.5 mg to about 20 mg ergothioneine; and
4) about 27.5 μg to about 50 μg selenium.

In a further embodiment, there is provided a composition comprising:
1) about 250 mg urolithin A;
2) about 150 mg phosphatlidylserine;
3) about 12.5 mg ergothioneine; and
4) about 27.5 μg selenium.

A composition of the invention may also comprise one or more brain-essential nutrients. Brain essential nutrients include Vitamin B6, Vitamin B9, Vitamin B12, magnesium salts, folic acid and L-methylfolate. The compositions of the invention may contain all of Vitamins B6, B9 and B12. The compositions of the invention may contain, in particular, L-methylfolate. Magnesium may, for example, be provided in the form of a citrate or threonate salt or as part of a magnesium phospholipid complex.

In a further embodiment, a composition of the invention, comprises a one or more brain-essential nutrients comprising Vitamin B6, Vitamin B9 and/or Vitamin B12.

In an embodiment of the invention, there is provided a composition comprising:
(a) a compound of formula (I), or a salt, prodrug, or pharmaceutically acceptable salt thereof;
(b) phosphatidylserine; and
(c) L-methylfolate.

In an embodiment of the invention, there is provided a composition comprising:
(a) a compound of formula (I), or a salt, prodrug, or pharmaceutically acceptable salt thereof;
(b) ergothioneine; and
(c) L-methylfolate.

In an embodiment of the invention, there is provided a composition comprising:
(a) a compound of formula (I), or a salt, prodrug, or pharmaceutically acceptable salt thereof;
(b) selenium; and
(c) L-methylfolate.

There is further provided a composition comprising:
(a) a compound of formula (I), or a salt, prodrug, or pharmaceutically acceptable salt thereof;
(b) phosphatidylserine;
(c) ergothioneine; and
(d) L-methylfolate.

There is further provided a composition comprising:
(a) a compound of formula (I), or a salt, prodrug, or pharmaceutically acceptable salt thereof;
(b) phosphatidylserine;
(c) selenium; and
(d) L-methylfolate.

There is further provided a composition comprising:
(a) a compound of formula (I), or a salt, prodrug, or pharmaceutically acceptable salt thereof;
(b) ergothioneine;
(c) selenium; and
(d) L-methylfolate

A composition of the invention may also comprise medium chain triglycerides and/or ketones. Ketones may include: β-Hydroxybutyrate (BHB) or BHB esters. The daily dose of ketone body (or salt, prodrug, metabolite or derivative thereof) provided by compositions of the invention may be in the range of about 20 mg to about 5000 mg, for example about 20 mg to about 4000 mg, for example about 200 mg to about 4000 mg, for example about 20 mg to about 3000 mg, for example about 20 mg to about 2000 mg, for example about 100 mg to about 1000 mg, for example about 100 mg to about 800 mg, for example about 200 mg to about 600 mg, for example about 200 mg to about 400 mg, for example about 200 mg to about 300 mg, for example about 250 mg. The daily intake of ketone body (or salt, prodrug, metabolite or derivative thereof) may be provided as a single serving, or may be divided between multiple servings. Alternatively, the daily dose of ketone body (or salt, prodrug, metabolite or derivative thereof) provided by compositions of the invention may be in the range of about 2 g to about 50 g, for example about 5 g to about 30 g, for example about 10 g to about 20 g.

Typically, a composition of the invention comprises from 100 mg to 2500 mg of the compound of formula (I). As described in further detail below, a preferred compound of formula (I) is Urolithin A. That is to say that a composition of the invention may comprise from 100 mg to 2500 mg of Urolithin A.

In a further embodiment, a composition of the invention comprises about 50 milligrams to about 300 milligrams phosphatidylserine, for example about 100 milligrams to about 200 milligrams phosphatidylserine, for example, 120 milligrams phosphatidylserine.

In a further embodiment, a composition of the invention comprises about 2 to about 50 milligrams ergothioneine, for example about 5 to about 35 milligrams ergothioneine, for example 5 to 25 milligrams ergothioneine, for example 25 milligrams ergothioneine.

In a further embodiment, a composition of the invention comprises about 500 to about 1500 milligrams taurine.

In a further embodiment, a composition of the invention comprises about 100 milligrams to about 200 millgrams L-theanine.

In a further embodiment, a composition of the invention comprises about 200 micrograms to about 600 micrograms alpha GPC.

In a further embodiment, a composition of the invention comprises about 100 micrograms to about 400 micrograms huperzine-A. Huperzine-A may be provided as a plant extract comprising huperzine-A.

In a further embodiment, a composition of the invention comprises about 15 micrograms to about 100 micrograms selenium, for example about 25 micrograms to about 75 micrograms selenium, for example about 30 micrograms to about 50 micrograms selenium, for example 50 micrograms selenium.

In a further embodiment, a composition of the invention comprises about 200 micrograms to about 500 micrograms Vitamin B12. In a further embodiment, a composition of the invention comprises about 10 milligrams to about 20 milligrams Vitamin B6. In a further embodiment, a composition of the invention comprises about 400 micrograms to about 800 micrograms folate.

### Compositions Comprising an Agent for Improving Immune Health

In an embodiment of the invention, there is provides a composition comprising:
(a) a compound of formula (I), or a salt, prodrug, or pharmaceutically acceptable salt thereof; and
(b) one or more agents selected from agents for improving immune health.

In an embodiment of the invention, there is provides a composition comprising:
(a) a compound of formula (I), or a salt, prodrug, or pharmaceutically acceptable salt thereof; and
(b) one or more agents selected from agents for improving immune health, comprising quercetin.

In a further embodiment of the invention, there is provides a composition comprising:
(a) a compound of formula (I), or a salt, prodrug, or pharmaceutically acceptable salt thereof; and
(b) one or more agents selected from agents for improving immune health, comprising:
   i). quercetin; and
   ii). Elderberry.

An agent for improving immune health may, for example, be selected from:
(i) Elderberry or an extract thereof;
(ii) Selenium; and
(iii) a zinc salt.

In a further embodiment of the invention, there is provides a composition comprising:
(a) a compound of formula (I), or a salt, prodrug, or pharmaceutically acceptable salt thereof; for example, urolithin A; and
(b) one or more agents selected from agents for improving immune health, comprising:
   a. quercetin;
   b. elderberry; and
   c. a zinc salt and/or a selenium salt, for example, a zinc salt and a selenium salt.

In an embodiment, compositions of the invention also comprise quercetin. In one embodiment, compositions of the invention comprise quercetin in the range about 100 mg to about 1000 mg, for example, about 100 mg to about 250 mg or about 250 mg to about 500 mg or about 500 mg to about 1000 mg.

In a further embodiment, compositions of the invention comprise L-theanine. In one embodiment, compositions of the invention comprise L-theanine in the range about 110 mg to about 900 mg.

In a further embodiment compositions of the invention comprise ashwagandha. In one embodiment, compositions of the invention comprise ashwagandha in the range about 250 mg to about 600 mg.

In a further embodiment compositions of the invention comprise a cannabidiol (CDB). In one embodiment, compositions of the invention comprise cannabidiol in the range about 110 mg to about 900 mg.

In a further embodiment, compositions of the invention comprise melatonin. In one embodiment, compositions of the invention comprise melatonin in the range about 0.5 mg to about 10 mg.

In a further embodiment, compositions of the invention comprise ginkgo, and/or components of ginkgo extracts, such as flavonoids, polyphenols and/or terpenoids, for example, EGb 761, kaempferol and ginkgolides (for example, Ginkgolides A, B, C, J and/or M).

In a further embodiment, compositions of the invention comprise lion's mane. Lion's mane are white mushrooms which resemble a lion's mane. For example, Hericium erinaceus is one of the most widely available. In one embodiment, compositions of the invention comprise lion's mane in the range about 500 mg to about 3000 mg.

In a further embodiment, compositions of the invention comprise citicoline. In one embodiment, compositions of the invention comprise citicoline in the range about 100 mg to about 1000 mg.

In a further embodiment of the invention, there is provides a composition comprising:
(a) a compound of formula (I), or a salt, prodrug, or pharmaceutically acceptable salt thereof; for example, urolithin A;
(b) one or more agents that enhance neurotransmission (for example a nootropic, for example ergothioneine, huperzine, aGPC, taurine or L-theanine);
(c) one or more brain protective agents (for example phosphatidylserine or phosphatidylcholine; and/or melatonin; and
(d) L-theanine and/or ashwagandha.

In a further embodiment of the invention, there is provides a composition comprising:
(a) a compound of formula (I), or a salt, prodrug, or pharmaceutically acceptable salt thereof, for example, urolithin A; and
(b) L-theanine and/or ashwagandha.

The composition may also comprise a further mitophagy activator and/or a mitochondrial function boosting agent (for example a NAD booster, resveratrol, CoQ10 or pyrroloquinoline quinone (PQQ)). In one embodiment, compositions of the invention comprise resveratrol in the range about 100 mg to about 2000 mg, for example, about 100 mg to about 250 mg or about 250 mg to about 500 mg. In one embodiment, compositions of the invention comprise PQQ in the range about 0.5 mg to about 20 mg. In one embodiment, compositions of the invention comprise CoQ10 in the range about 50 mg to about 500 mg.

In an embodiment of the invention, there is provided a composition comprising:
(a) a compound of formula (I), or a salt, prodrug, or pharmaceutically acceptable salt thereof; and
(b) an immune health improving agent.

In an embodiment of the invention, there is provided a composition comprising:
(a) a compound of formula (I), or a salt, prodrug, or pharmaceutically acceptable salt thereof; and
(b) Elderberry or an extract thereof.

In an embodiment of the invention, there is provided a composition comprising:
(a) a compound of formula (I), or a salt, prodrug, or pharmaceutically acceptable salt thereof; and
(b) Selenium.

In an embodiment of the invention, there is provided a composition comprising:
(a) a compound of formula (I), or a salt, prodrug, or pharmaceutically acceptable salt thereof; and
(b) a zinc salt.

Examples of zinc salts include zinc oxide, zinc sulphate, zinc picolinate, zinc citrate, zinc acetate, zinc glycerate, zinc gluconate, zinc bisglycinate, zinc orotate, and zinc monomethionine. For example, zinc gluconate, zinc picolinate and zinc bisglycinate. In a further embodiment, the zinc salt is selected from one or more salts selected from zinc picolinate, zinc citrate, zinc gluconate, zinc bisglycinate, zinc monomethionine and zinc acetate. In one embodiment, the zinc salt is zinc picolinate. The zinc has been found to have has a synergistic effect with a compound of formula (I) and in particular with urolithin A.

In a further embodiment, a composition of the invention comprises about 2 milligrams to about 50 milligrams elemental zinc, for example about 2.5 mg to about 50 mg, 5 mg to about 50 mg, about 10 mg to about 40 mg zinc, about 30 mg to about 50 mg, about 5 milligrams to about 20 milligrams zinc, about 5 mg to about 10 mg, for example about 2.5 mg, about 2.5 mg, 10 milligrams zinc, about 15 mg zinc, about 25 mg zinc, about 50 mg zinc. Zinc is included in the form of a salt for example, zinc picolinate, zinc citrate, zinc acetate, zinc glycerate, zinc sulphate, Zinc bisglycinate, and zinc monomethionine. Preferably, the zinc bysglycinate (27% zinc) is used.

In a further embodiment of the invention, there is provides a composition further optionally comprising one or more additional components selected from ginseng, echinacea, immuse, bromelain, lactoferrin, colostrum, β-glucan, lysine and arginine.

In an embodiment of the invention, there is provided a composition comprising:
(a) a compound of formula (I), or a salt, prodrug, or pharmaceutically acceptable salt thereof; for example, urolithin A, or a salt, prodrug, or pharmaceutically acceptable salt thereof;
(b) elderberry, or an extract thereof;
(c) a salt of zinc;
(d) selenium;
(e) optionally quercetin;
(f) optionally ginseng;
(g) optionally one or more ginseng-derived compounds or a ginseng extract; and/or one of more notoginseng-derived compounds or a notoginseng extract;
(h) optionally echinacea;
(i) optionally immuse;
(j) optionally bromelain;
(k) optionally lactoferrin;
(l) optionally colostrum;
(m) optionally β-glucan;
(n) optionally lysine; and
(o) optionally arginine

In embodiments of the invention, ginseng-derived compounds, include ginsenosides, for example, Rb1, Rb2, Rg3, Rh2, Rh3, Rg1, Rg2, and/or Rh1. In embodiments of the invention, notoginseng-derived compounds include notoginsenosides, for example, R1, Rt, R2, R3, R4, and/or R6.

In a further embodiments, compositions of the invention further comprise a senolytics.

In one embodiment a senolytic is selected from one of more of the group apigenin, fisetin, curcumin and luteolin, for example, fisetin.

In an embodiment of the invention, there is provided a composition comprising:
(a) a compound of formula (I), or a salt, prodrug, or pharmaceutically acceptable salt thereof, for example urolithin A, and
(b) a senolytic is selected from one of more of the group apigenin, fisetin, curcumin and luteolin, for example, fisetin.

Typically, a composition of the invention comprises from about 125 mg to about 2500 mg, for example, about 200 mg to about 2500 mg, such as about 500 mg to about 2000 mg of the compound of formula (I). As described in further detail below, a preferred compound of formula (I) is Urolithin A. That is to say that a composition of the invention may comprise from about 125 mg to 2500 mg of Urolithin A. For example, about 125 mg, about 250 mg, 500 mg, about 1000 mg, about 1500 mg or about 2000 mg urolithin A.

In a further embodiment, a composition of the invention comprises about 50 milligrams to about 400 milligrams of Elderberry or Elderberry extract, for example about 100 milligrams to about 300 milligrams Elderberry or Elderberry extract, for example about 200 milligrams Elderberry or Elderberry extract.

In a further embodiment, a composition of the invention comprises about 5 to about 20 milligrams zinc, for example about 5 to about 15 milligrams zinc, for example, about 5 mg to about 10 mg, for example 10 milligrams zinc. The zinc is typically provided in the form of a salt, for example as zinc oxide, zinc sulphate, zinc acetate, zinc citrate, zinc gluconate, zinc picolinate, zinc orotate, and zinc bisglycinate. The mass of zinc stated is the mass of the zinc itself in the salt.

In a further embodiment, a composition of the invention comprises about 30 micrograms to about 100 micrograms selenium, for example about 25 micrograms to about 75 micrograms selenium, for example about 30 micrograms to about 50 micrograms selenium, for example 50 micrograms selenium. The selenium is typically provided as a salt, or as part of a seleno-amino acid, selenopeptide, or selenoprotein, for example, selenomethionine. As a salt, selenium is typically in the form of, for example, sodium selenite, sodium selenate, potassium selenite, and potassium selenate. As a seleno-amino acid, selenium can be incorporated into sulphur-containing amino acids, for example methionine and cysteine, where the selenium replaces the sulphur. These seleno-amino acids can be incorporated into peptide or protein chains, where the peptide or protein chains are referred to as selenopeptides or selenoproteins respectively. The mass of selenium stated is the mass of the selenium itself in the salt. Compositions may also contain selenium in a form organically bound in an inactive yeast (for example, Saccharomyces cerevisiae), for example, SelenoExcell® (Cypress, Madera, California, USA) [https://cypressingredients.com/selenoexcell/].

In a further embodiment, a composition of the invention comprises about 100 milligrams to about 400 milligrams of Quercetin, for example about 100 milligrams to about 300 milligrams quercetin, for example 250 milligrams quercetin.

In one embodiment, the selenium is provided as an organically bound form of selenium, for example, selenomethionine, selenocysteine, and/or methyl-selenocysteine

### Compounds of Formula (I) (Urolithins)

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in the below table, with reference to structure

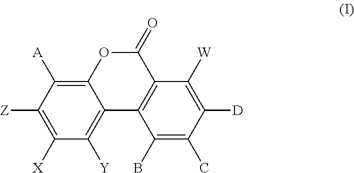

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the combinations of the invention.

In one aspect of a combinations of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

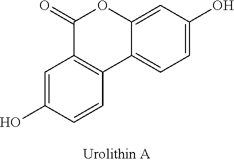 [SMILES: O=c1oc2cc(O)ccc2c2ccc(O)cc12]

According to one embodiment there is provided a combination, composition, use or method of the invention wherein the compound of formula (I) is urolithin A.

According to one embodiment there is provided a combination, composition, use or method of the invention wherein the compound of formula (I) is urolithin B.

According to one embodiment there is provided a combination, composition, use or method of the invention wherein the compound of formula (I) is urolithin C.

According to one embodiment there is provided a combination, composition, use or method of the invention wherein the compound of formula (I) is urolithin 0.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, di-isopropyl-, triethyl-, tributyl- or dimethyl-propylamine, or a mono-, di- or trihydroxy lower alkylamine, for example mono-, di- or triethanolamine.

### Urolithin Administration/Dosage Regimes

The administration of compositions of the present invention preferably involves oral administration of a urolithin of formula (I) or salt thereof to a subject in a daily amount in the range of 1.7 to 6.0 mmol per day, for example, from about 1.7 to about 2.7 mmol per day, or from about 2.8 to about 6.0 mmol per day. As discussed below, administration is preferred in the range 250 mg to 2000 mg urolithin A (which corresponds to about 1.1 to 8.8 mmol), for example 125 mg to 1500 mg, such as 250 mg to 1500 mg, for example, 250 mg to 1250 mg, or 250 mg to 1000 mg, which results in a surprisingly good pharmacokinetic profile. In one embodiment the dose is 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
125 mg once or twice a day
250 mg once or twice a day;
500 mg once or twice a day;
750 mg once or twice a day;
1000 mg once or twice a day;
1250 mg once or twice a day; or
1500 mg once or twice a day

The uses and methods of the present invention involve daily administration of the compound of formula (I) or salt thereof, or of a composition comprising the compound or salt. In some embodiments, the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

The uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4, 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from 400 to 600 mg/day or 400 to 600 mg twice per day. In a preferred embodiment the method involves administration of urolithin A in an amount in the range of from 450 to 550 mg, more preferably approximately 500 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

In other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

Dosage regimes which combine two different doses of urolithin A, for example, a 250 mg dose and a 125 mg dose, a 500 mg dose and 250 mg dose or a 500 mg dose and a 1000 mg dose may be advantageous. For example, a twice daily dosage regime which combines a first dose of for example 1000 mg and a second dose several hours later of for example, 500 mg. Said 500 mg dose may be 6-18 hours after the 1000 mg dose, for example 8-12 hours after the 1000 mg dose. For example, about 12 hours after the 1000 mg dose. Thus, according to a further aspect of the invention there is provided the treatment of a disease, disorder or condition with a compound of Formula (I) which comprises a twice daily dosage regime comprising a first dose of 1000 mg, followed by a second dose of 500 mg wherein the two doses are separated by 6-18 hours.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided a composition wherein the compound of formula (I) is administered at a dose of about 4.5-18 mg/kg.

### Additional/Combination Therapy

Compositions of the invention may comprise one of more further active agents, for use in the treatment or prevention of diseases, disorders or conditions.

Any active agent which is known to be useful, or which has been used or is currently being used for the treatment or prevention of diseases, disorders or conditions can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used for the treatment or prevention of diseases, disorders or conditions associated with brain health and/or immune health.

### Uses

Compositions of the invention can be used for both therapeutic and non-therapeutic uses, finding use in the treatment of various diseases as well as health conditions not considered to be a disease. In particular, disease and non-disease health conditions may be characterised by an inadequate mitochondrial activity. Therefore, according to one embodiment of the invention there is provided a composition of the invention for use as a medicament for the treatment of a disease disorder or condition. According to a further embodiment of the invention there is provided a non-therapeutic method of use of a composition of the invention.

The compositions of the invention can be used as a dietary supplement, as a food product, a functional food and as a medical food. The compositions of the invention can be used for food for special medical purposes.

Compositions of the invention find utility in the treatment of disease, disorders and conditions. Therefore, according to a further aspect of the invention, there is provided a composition of the invention for use as a medicament for the treatment of a disease disorder or condition.

According to a further aspect of the invention there is provided a composition, of the invention for use in the treatment, prevention or management of a mitochondria-related condition associated with altered mitochondrial function or reduced mitochondrial density.

According to a further aspect of the invention there is provided a non-therapeutic method for preventing or managing a mitochondria-related condition associated with altered mitochondrial function (such as decreased mitochondrial function including due to the presence of fewer mitochondria and/or the presence of damaged mitochondria) or reduced mitochondrial density, comprising administration of an effective amount of a composition of the invention.

According to a further aspect of the invention there is provided a combination or composition of the invention for use in the treatment, prevention or management of a mitochondria-related disease associated with altered mitochondrial function or reduced mitochondrial density.

According to a further aspect of the invention there is provided a composition, of the invention for use in the treatment, prevention or management of a mitochondria-related disease.

According to a further aspect of the invention there is provided a combination or composition of the invention for use in increasing or maintaining mitochondrial function.

According to a further aspect of the invention there is provided a non-therapeutic method of increasing or maintaining mitochondrial function, comprising administration of an effective amount of a composition of the invention.

### Therapeutic Uses

### Neurological Disorders

According to one embodiment of the invention, there is provided a composition of the invention for use in the treatment of neurological disorders, including neurodegenerative disorders. Examples of neurological disorders include: cognitive decline, memory decline, anxiety, depression, epilepsy, stroke, amyotrophic lateral sclerosis, dementia (e.g. vascular dementia and Alzheimer's disease), Parkinson's disease. and Huntington disease.

According to a further embodiment of the invention, there is provided a composition of the invention for use in the prophylaxis or treatment of a disease state initiated or characterized (i) by a decline in cognitive function; or (ii) by mood disturbances. Such disease states can include, without limitation, neurodegenerative disease, cognitive disorder, mood disorder and anxiety disorder.

An embodiment of the invention is a method of improving cognitive function. The method includes the step of administering to a subject in need thereof an effective amount of a urolithin or a precursor thereof, to improve cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

According to a further embodiment, there is provided a composition of the invention for use for protecting against brain oxidative stress.

According to a further embodiment, there is provided a composition of the invention for use for reducing brain inflammation.

According to a further embodiment, there is provided a composition of the invention for use for treating mood disorders, stress and/or anxiety disorders.

### Immune Function

In a further embodiment of the invention there is provided the use of a composition of the invention for supporting and/or enhancing immune function during or after cancer treatment.

In a further embodiment of the invention there is provided the use of a composition of the invention for supporting and/or enhancing immune function remission after cancer treatment.

In a further embodiment of the invention there is provided the use of a composition of the invention for supporting and/or enhancing immune function during and after hospital admissions.

### Immune Health

In a further embodiment of the invention there is provided the use of a composition of the invention for maintaining or enhancing immune health.

In a further embodiment of the invention there is provided the use of a composition of the invention for reducing or slowing inflammaging.

In a further embodiment of the invention there is provided the use of a composition of the invention for slowing immune aging.

In a further embodiment of the invention there is provided the use of a composition of the invention for reducing immune cell aging.

In a further embodiment of the invention there is provided the use of a composition of the invention for boosting immune function.

In a further embodiment of the invention there is provided the use of a composition of the invention for supporting a healthy immune system.

In a further embodiment of the invention there is provided the use of a composition of the invention for prevention or management of colds and/or flu.

In a further embodiment of the invention there is provided the use of a composition of the invention for the treatment of infection.

In a further embodiment of the invention there is provided the use of a composition of the invention for improving vaccination response.

In a further embodiment of the invention there is provided the use of a composition of the invention for enhancing an immune response against infection.

In a further embodiment of the invention there is provided the use of a composition of the invention for protecting against infection, for example, protection against colds and flu.

In a further embodiment of the invention there is provided the use of a composition of the invention for immune support.

In a further embodiment of the invention there is provided the use of a composition of the invention for reducing the risk of respiratory tract infections (for example, colds, flu or pneumonia).

In a further embodiment of the invention there is provided the use of a composition of the invention for increasing the rate of recovery from respiratory tract infections (for example, colds, flu or pneumonia).

In a further embodiment of the invention there is provided the use of a composition of the invention for increasing CD8 positive T-cells, for example, naïve CD8-positive T-cells.

In a further embodiment of the invention, there is provided a composition of the invention for maintaining or enhancing immune function, wherein the maintaining or enhancing of immune function comprises one or more of the following:
(a) reducing the risk of/protecting against respiratory tract infections (for example, colds, flu or pneumonia);
(b) increasing the rate of recovery from respiratory tract infections (for example, colds, flu or pneumonia);
(c) improving vaccination response;
(d) enhancing an immune response against infection;
(e) treating or for preventing immunosenescence;
(f) improving the mitochondrial function in immune cells;
(g) reducing or slowing inflammaging;
(h) increasing CD8 positive T-cells, for example, naïve CD8-positive T-cells.
(i) Slowing, reversing, managing immune aging and/or aging of immune cells; and/or
(j) Improving metabolic function of immune cells.

### Aging/Healthspan

According to a further embodiment of the invention, there is provided a composition of the invention for use in the increasing or prolonging of healthspan. Healthspan can be defined as the part of a person's life during which they are generally in good health. In a further embodiment of the invention, there is provided a composition of the invention, for slowing aging.

According to a further embodiment of the invention, there is provided a composition of the invention for use for delaying cellular senescence.

### Treatments:

The composition of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

### Non-Therapeutic Uses

Compositions of the invention find use as nutritional supplements, functional foods, medical nutrition and equivalents. These include treatments of non-therapeutic conditions, i.e. treatment of conditions in generally healthy subjects.

### Muscle Function, Performance, Endurance

The composition finds use in the management normal physiological function in healthy individuals of conditions characterised by poor physical performance, impaired endurance capacity, and impaired muscle function. Compositions of the invention may improve physical performance in individuals with a disease, including young and elderly individuals. Compositions of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Compositions of the invention further provide for the improvement of endurance capacity. The endurance capacity refers to the time to fatigue when exercising at a constant workload, generally at an intensity <80% VO₂max. Compositions of the invention may improve endurance capacity in individuals with a disease, including young and elderly individuals. Compositions of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a composition to improve, maintain or reduce the loss of muscle function. Compositions of the invention may improve, maintain or reduce the loss of muscle function in individuals with a disease, including young and elderly individuals. Compositions of the invention may improve, maintain or reduce the loss of muscle function in healthy individuals, including athletes, non-athletic individuals, sedentary individuals and the elderly. For example, compositions of the invention may increase muscle strength as evidenced by the improvement of performing a physical activity, such as an exercise, for example, increased ability to lift weights or increased hand grip strength. Also, compositions of the invention may improve muscle structure, for example by increasing or maintaining muscle mass in conditions of normal muscle function, declining muscle function or impaired muscle function.

This invention further provides a composition to improve the physical performance or endurance capacity as perceived by the individual. For example, by the reduction of in perceived exertion or effort during exercise or an activity as determined using a self-reported questionnaire.

### Muscle Performance

The composition of the invention is useful in enhancing muscle performance. The invention thus provides a composition of the invention for use in enhancing muscle performance. The invention also provides a method of enhancing muscle performance by administering to a subject an effective amount of a composition of the invention. Administration can be self-administration. The enhanced muscle performance may be one or more improved muscle function, improved muscle strength, improved muscle endurance and improved muscle recovery.

The composition of the invention can thus be used in a method of improving physical endurance (e.g., ability to perform a physical task such as exercise, physical labor, sports activities), inhibiting or retarding physical fatigue, enhancing working capacity and endurance, reducing muscle fatigue, enhancing cardiac and cardiovascular function.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The composition of the invention may be used in enhancing muscle performance by administering a composition of the invention to a subject who is frail or pre-frail.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

### Brain Health

According to a further embodiment of the invention there is provided the use of a composition of the invention for maintaining or enhancing brain health.

According to a further embodiment of the invention there is provided the use of a composition of the invention for promoting healthy brain function.

According to a further embodiment, there is provided a composition of the invention for use for optimising brain health.

According to a further embodiment, there is provided a composition of the invention for use to improve, protect, and maintain brain function and cognition.

According to a further embodiment, there is provided a composition of the invention for use for enhancing mental alertness, for example, mental accuracy and clearer/sharper thinking.

According to a further embodiment, there is provided a composition of the invention for use for increasing psychomotor speed and/or reaction time.

According to a further embodiment, there is provided a composition of the invention for use for enhancing/improving memory.

According to a further embodiment, there is provided a composition of the invention for use for enhancing/improving learning.

According to a further embodiment, there is provided a composition of the invention for use for enhancing/improving reasoning, for example, promoting mental processing.

According to a further embodiment, there is provided a composition of the invention for use for enhancing/improving focus, for example, concentration, complex attention and/or sustained attention.

According to a further embodiment, there is provided the use of a composition of the invention for stress relief and/or anxiety relief.

According to a further embodiment, there is provided the use of a composition of the invention for treating stress and/or anxiety.

According to a further embodiment, there is provided the use of a composition of the invention for treating depression.

According to a further embodiment, there is provided the use of a composition of the invention for improving mood.

According to a further embodiment, there is provided the use of a composition of the invention for enhancing sleep and/or relaxation.

According to a further embodiment, there is provided the use of a composition of the invention for one or more of the following:
1) Supporting mental acuity;
2) Supporting verbal and nonverbal memory;
3) supporting verbal fluency;
4) Supporting cognitive function/health; and/or
5) Sharpening attention.

According to a further embodiment, there is provided the use of a composition of the invention for one or more of the following:
1) Enhancing mental acuity;
2) Enhancing verbal and nonverbal memory;
3) Enhancing verbal fluency;
4) Enhancing cognitive function/health; and/or
5) Enhancing attention.

According to a further embodiment, there is provided the use of a composition of the invention for one or more of the following:
1) Reducing exhaustion;
2) Reducing feelings of exhaustion;
3) Supporting energy;
4) Supporting vitality; and/or
5) Supporting feeling motivated.

According to a further embodiment, there is provided the use of a composition of the invention for one or more of the following:
1) Supporting restful sleep;
2) Supporting sleep onset;
3) Supporting feeling well rested; and/or
4) Supporting healthy sleep.

According to a further embodiment, there is provided the use of a composition of the invention for one or more of the following:
1) Supporting concentration;
2) Supporting attention; and/or
3) Supporting short term memory According to a further embodiment, there is provided the use of a composition of the invention for one or more of the following:
1) Enhancing mental sharpness;
2) Protecting mental sharpness;
3) enhancing concentration;
4) enhancing attention; and/or
5) enhancing short term memory.

According to a further embodiment, there is provided the use of a composition of the invention for improving one or more of the following:
(1) memory disturbances;
(2) loss of focus;
(3) brain fog;

According to a further embodiment, there is provided the use of a composition of the invention for promoting peak cognitive function.

### Animals

Compositions of the invention are suitable for other mammals in addition to humans, scaled appropriately to the size of the non-human mammal. For example, scaled according to the following table;

| | | | To convert human | |
|---|---|---|---|---|
| | | | dose in mg/kg to AED | |
| | Reference | To convert dose | in mg/kg, either | |
| | body | in mg/kg to dose | Multiply | Divide |
| | weight | in mg/m2, divide | human | human |
| Species | (kg) | by km | dose by | dose by |
| Mouse | 0.02 | 3 | 12.3 | 0.081 |
| Hamster | 0.08 | 5 | 7.4 | 0.135 |
| Rat | 0.15 | 6 | 6.2 | 0.162 |
| Ferret | 0.3 | 7 | 5.3 | 0.189 |
| Guinea Pig | 0.4 | 8 | 4.6 | 0.216 |
| Rabbit | 1.8 | 12 | 3.1 | 0.324 |
| Cat | 2 | 11.7 | 3.2 | 0.316 |
| Monkey | 3 | 12 | 3.1 | 0.324 |
| Dog | 10 | 20 | 1.9 | 0.541 |
| Marmoset | 0.35 | 6 | 6.2 | 0.162 |
| Squireel | 0.6 | 7 | 5.3 | 0.189 |
| Monkey | | | | |
| Baboon | 12 | 20 | 1.9 | 0.541 |
| Micro pig | 12 | 27 | 1.4 | 0.73 |
| Mini pig | 40 | 35 | 1.1 | 0.946 |

Therefore, according to a further aspect of the invention there is provided a composition for use in a non-human mammal wherein the dose is scaled according to the table above.

For example, compositions of the invention for humans, dogs and cats comprise components in dose ranges as listed in Tables 7-11.

### Compositions

The uses and methods of the present invention preferably involve oral administration of the compositions of the invention. Any suitable oral composition may be used. Accordingly, the use of a range of compositions which are suitable for oral administration, is envisaged. Thus in some embodiments, the composition is administered in the form of an oral composition and one or more excipients suitable for oral administration. Oral compositions may comprise compositions having the form of a pill, tablet, capsule, caplet, lozenge, pastille, granules, powder for suspension, oral solution, oral suspension, oral emulsion, syrup, or the like.

In a further embodiment of the invention, the composition is administered by any means known to the skilled person for administration such as, intramuscular, sublingual, cutaneous, inhalation and auricular. Oral administration is preferred.

Compositions may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability.

At certain levels of dilution, a drink may need to be shaken before the subject drinks it, so as to maintain an even suspension of the active ingredient.

The compositions of the present invention described herein may be provided as a soft gel (for example in a capsule having a shell), as a gummy, as a powder as a tablet, as a lotion or as a cream.

Compositions may comprise shells comprising non-animal components, for example, vegetarian softshells.

In some preferred embodiments, the use or method comprises administration of the compound of formula (I) or salt thereof (e.g. urolithin A), with a defined particle size distribution. A defined particle size distribution enables the compound of formula (I) to disperse or dissolve more rapidly. A defined particle size distribution can be achieved by methods established in the art, for example compressive force milling, hammer milling, universal or pin milling, or jet milling (for example spiral jet milling or fluidised-bed jet milling) may be used. Jet milling is especially suitable. Alternatively, chemical processes for the preparation of a compound of formula (I) may produce a compound of formula (I) of the required particle size distribution. If a compound of formula (I) with a defined particle size distribution is used, then preferably the compound has a D₅₀ size of under 100 μm-that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm.

Preferably, the compound has a D₉₀ size of under 100 μm. More preferably, the compound has a D₉₀ size of under 75 μm, for example under 50 μm, for example under 25 μm, for example under 20 μm, for example under 15 μm. The compound preferably has a D₉₀ in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a Do in the range 8.2 to 16.0 μm, a D₅₀ in the range 2.8 to 5.5 μm and a D₁₀ in the range 0.5 to 1.0 μm.

In a further embodiment, the compound of formula (I) or salt thereof has a size distribution selected from one of the following:
(i) D₅₀ size in the range 0.5 to 50 μm and a Do size in the range 5 to 100 μm,
(ii) the compound has a D₉₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a Do size under 50 μm and a D₉₀ size under 75 μm;
(v) the compound of Formula (I) has a Do size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉ size under 15 μm; or
(vi) the compound of Formula (I) has a D₅₀ size of 10 μm and a D₉₀ size of 20 μm.

For the avoidance of doubt, compounds in compositions of the invention may be formulated in the same composition or formulated in separate compositions for simultaneous, separate or sequential administration.

### Kits

Also within the scope of the present invention are kits, comprising composition of the invention. Kits typically include a label indicating the intended use of the contents of the kit and instructions for use. The term “label” includes any writing, or recorded material supplied on or with the kit, or which otherwise accompanies the kit.

The invention further provides kits comprising:
(a) a composition of the invention; and
(b) a container, or containers, for containing said agents; and
(c) optionally instructions for simultaneous, separate or sequential administration.

### Preparation of Compositions of the Invention

In a further embodiment, there is provided a process for preparing a composition of the invention, which comprises, mixing
(a) a compound of formula (I), or a salt, prodrug, or pharmaceutically acceptable salt thereof, for example, urolithin A;
(b) the further active ingredient(s), and
(c) one or more excipients, for example, one of more excipients suitable for oral administration.

The term ‘about’ refers to a tolerance of ±20% of the relevant value, for example ±15% of the relevant value, such as ±10% of the relevant value or ±5% of the relevant value.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing

The term ‘imflammaging’ refers to chronic, low-grade systemic inflammation that develops with increasing age, even in the absence of infection. Imflammaging contributes to cognitive decline and neurodegenerative diseases (e.g., Alzheimer's Disease).

The term ‘nootropic’ refers to a group of compounds, which improve human thinking, learning and memory. Examples of nootropic compounds include Huperzine A, aGPC, L-theanine, caffeine, taurine, ergothioneine, Meclofenoxate and Lions mane.

The term “pharmaceutically acceptable” means approved by a regulatory agency of the Federal or a state government or listed in the U.S. Pharmacopeia or other generally recognized pharmacopeia for use in animals, and more particularly in humans.

The term ‘senolytic’ refers to a group of molecules which selectively induce death of senescent cells.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a patient from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a patient in a single action such as where each compound are administered independently at substantially the same time or separately within time intervals that allow the compounds to show' a cooperative therapeutic effect.

The term “therapeutically effective amount” as used herein refers to the amount of a compound or compounds that, when administered, is sufficient to prevent the development of, or to relieve to some degree, one or more symptoms of the disease that it targets. The particular dose of each compound administered according to this invention will of course be determined by the particular conditions surrounding the case, including the compound administered, the route of administration, the particular condition being treated, as well as considerations such as age, weight and sex of the treated subject.

The invention will now be illustrated with respect to the following non-limiting examples

### EXAMPLES

### Example 1: Synergistic Combination of Low, Sub-Functional Doses of Ergothioneine with Sub-Functional Doses of Urolithin A

Chronic inflammation is a hallmark of brain aging (Gabuzda & Yankner (2013) Nature 297(7448), 197-8) and a key contributor to cognitive decline and the development of neurodegenerative diseases such as Alzheimer's and Parkinson's (Jin et al (2022) Int J Mol Sci 23(20): 12573; Jurcau et al (2024) Int J Mol Sci 25(19):10535).

Elevated levels of pro-inflammatory cytokines have been associated with reduced brain health. Interleukin 6 (IL-6), in particular, is not only a biomarker of systemic inflammation but also plays a direct role in neuroinflammation. Indeed, an age-dependent increase in IL-6 is occurring in brain tissues (Godbout & Johnson (2004) J Neuroimmunol 147(1-2), 141-4). Moreover, higher levels of IL-6 in older adults have been cross-sectionally and longitudinally associated with physical and cognitive impairment, as well as increased dementia risk and are associated with regionally increased rates of age-related cortical thinning. (McCarrey et al (2014) Transl Neurosci 5(1), 1-7) Therefore, targeting inflammation and reducing IL-6 levels is a promising strategy for preserving cognitive function and promoting brain health.

To evaluate the anti-inflammatory potential of compounds relevant to brain health, we employed the C2C12 mouse myoblast cell line as a model system. Although not derived from neural tissue, C2C12 cells are a well-established model for studying cellular stress responses, including inflammation, due to their robust and reproducible cytokine-induced inflammatory signaling. Importantly, several compounds known for their neuroprotective effects have shown consistent cell health benefits across cell types, including C2C12 cells. For instance, ergothieonine was shown to reduce IL-6 levels in stressed C2C12 cells (Laurenza et al (2008) Biofactors 33(4), 237-47) as well as in 3T3-L1 cells after stimulation with TNFα (Food Sci. Technol. Res., 17 (2), 103-110, 2011)—supporting the relevance of this model for screening brain health-promoting agents.

Moreover, C2C12 allows the investigation of systemic inflammation responses that can indirectly have an impact on brain health. Muscle-derived cytokines (myokines), including IL-6, can cross the blood-brain barrier and influence neuroinflammatory states. Studies suggest that systemic IL-6 levels are associated with decreased cognitive outcomes (McCarrey et al (2014) Transl Neurosci 5(1), 1-7).

In this example, we present data showing that the combination of Urolithin A and ergothioneine synergistically reduces IL-6 secretion in C2C12 cells exposed to a pro-inflammatory cytokine cocktail (IL-1β, TNF-α, and IFN-γ), highlighting their potential as a combinatorial intervention to mitigate neuroinflammation.

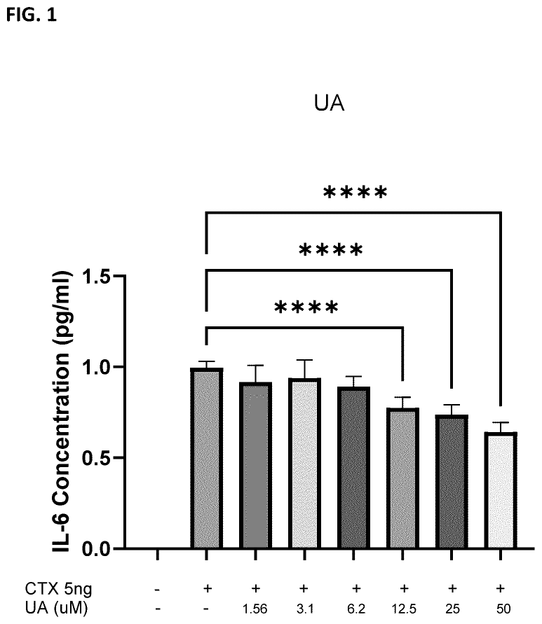

We first studied what is the lowest non-functional dose of Urolithin A in the context of cytokine-mediated muscle cell inflammation. For this, we exposed C2C12 myotubes to increasing doses of Urolithin A within a range of 1.56 uM to 50 mM for 24 hours, and then induced inflammation for an additional 24 hours by applying to cells a mix of pro-inflammatory cytokines (Cytomix, abbreviated CTX). One set of cells were left untreated, indicated by the first bar (CTX−, UA−). One set of cells were treated with CTX but not Urolithin A (second bar, CTX+, UA−). At the end of the study, we measure the concentration of the proinflammatory biomarker Interleukin 6 (IL-6) released in the cell culture media. As expected, CTX alone lead to a significant increase in release IL-6. The treatment of CTX inflamed cells with UA led to a dose dependent decrease in IL-6 release (FIG. 1). The first dose at which UA did not exert a significant reduction in IL-6 was 6.25 uM (FIG. 1). Therefore, 6.25 uM was selected as sub-functional dose for UA in the context of C2C12 inflammation reduction.

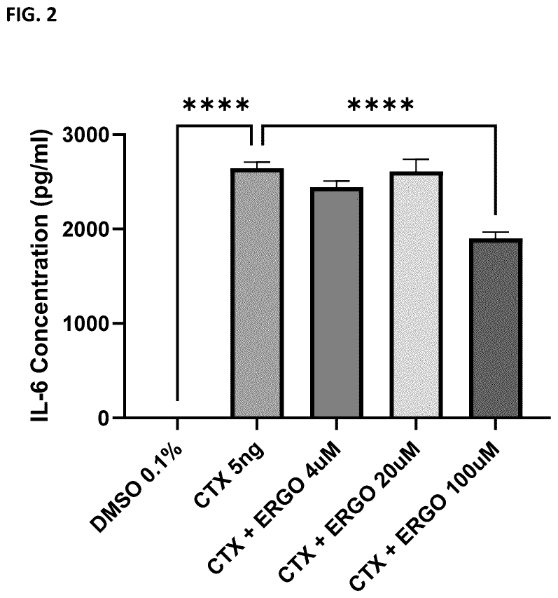

We performed the same dose range finding for the active compound ergothioneine (ERGO). The goal was to identify the most appropriate doses of ERGO to combine with UA to obtain superior anti-inflammatory responses. Based on the literature we used concentrations of ERGO at two low doses—potentially non-functional—i.e. 4 uM and 20 uM. And a higher dose of ERGO 100 uM were the compound showed biological effects in previous works. As for the UA experiment in FIG. 1, we treated C2C12 with ERGO at these three doses for 24 hours, followed by treatment with CTX for additional 24 hours. The 100 uM dose was the only one leading to a significant decrease in CTX-induced IL-6 secretion (FIG. 2). We therefore used the first sub-functional ERGO dose (20 uM) and first functional dose (100 uM) for testing in combination with UA.

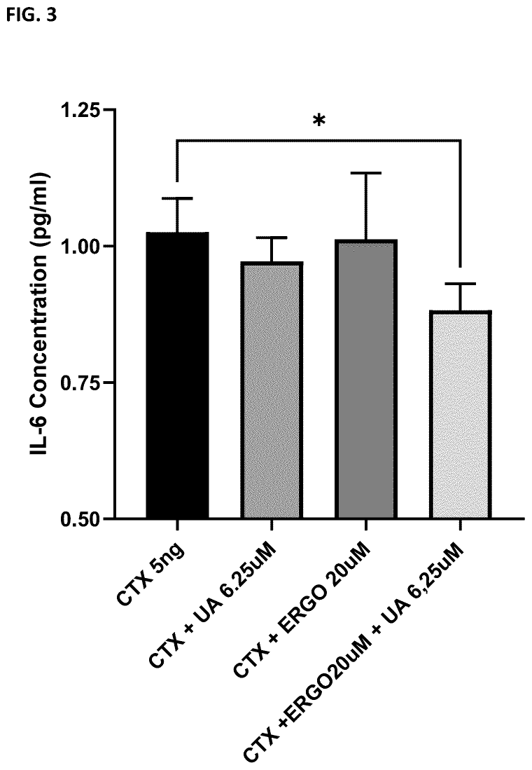

For this combination experiment, C2C12 were treated without CTX, with CTX alone and with CTX plus either UA alone (6.25 uM), ERGO alone (both 20 uM and 100 uM) and UA plus ERGO 20 uM and UA+ERGO 100 uM. We confirmed that the sub-functional doses of UA alone (6.25 uM) and ERGO alone (20 uM) did not significantly impact the release of IL-6. However, their combination lead to significant reduction in IL-6 release (−13.9%) and therefore anti-inflammatory status of the cells (FIG. 3 and Table 1). ERGO 100 uM induced a significant reduction in IL6. On the other hand, combining UA with an already functional dose of ERGO (100 uM) did not leave to a further decrease of IL-6. This indicates a specific dose range in which only certain doses of UA and ERGO combined together can significantly impact the cellular inflammatory status.

TABLE 1

| | Fold change of IL-6 | |
|---|---|---|
| Condition | (compared to CTX alone) | p. value |
| UA 6.25 uM | −5.2% | 0.6317 |
| ERGO 20 uM | +1.2% | 0.9913 |
| UA 6.25 uM + ERGO 20 uM | −13.9% | 0.0292 |

TABLE 2

| | Fold change of IL-6 | |
|---|---|---|
| Condition | (compared to CTX alone) | p. value |
| UA 6.25 uM | −5.2% | 0.3661 |
| ERGO 100 uM | −28% | <0.0001 |
| UA 6.25 uM + ERGO | −26.7% | <0.0001 |
| 100 uM | | |

### Methods (Example 1)

C2C12 Myoblasts purchased from the American Type Culture Collection (ATCC; ref. CRL-1772) were seeded at 8000 cells per well in a 96 well plate (Greiner) using a DMEM Glutamax 4.5 g D-glucose (Gibco, ref 31966021) supplemented with FBS 10% (PanBiotech, ref P190902), 1% PenStrep (Biowest, Nuaill6, France) and 1% Hepes (Biowest). Cells were then differentiated for 6 days using a differentiation medium (DMEM Glutamax 4.5 g D-glucose (Gibco, ref 31966021), 2% Horse serum (Thermofisher, ref 160501229) 1% PenStrep and 1% Hepes). All our media were sterilized with a 0.45 μm filter unit (VWR). After 4 days of the differentiation cells were treated for 24 h with 1) Urolithin A (UA), Ergothieoneine (ERGO, MedChem Express HY-N1914) or a combination of the two at the indicated doses. Cytokine mix (“cytomix”) was added at day 5 in addition of the previous treatment for another 24 h, cytokines were respectively used at 5 ng (mIFN-γ (Roche ref.11276905001), TNF-α (Peprotech ref. 300-01A) and IL-β (Proteintech ref. 200-011B)). The medium was collected and an ELISA for Interleukin-6 were performed following the instruction provided by the supplier (Proteintech ref. KE10007). Finally, the absorbance at a wavelength of 450 nm was measured using a microplate plate reader (Fluostar Optima, BMG Labtech).

### Example 2: Representative Powder or Gel Compositions with Benefits for Brain Health

For the avoidance of doubt representative compositions below may include other excipients to aid formulation or other active components.

2a. Formulation of Urolithin A with benefits for brain health

| | | |
|---|---|---|
| Component | Weight range | Weight |

| | | | | |
|---|---|---|---|---|
| Urolithin A | 250-2500 | mg | 500 | mg |
| Phosphatidylserine | 100-200 | mg | 125 | mg |
| Ergothioneine | 5-25 | mg | 25 | mg |
| Selenium | 30-100 | μg | 50 | μg |
| L-methylfolate | 5-15 | mg | 7.5 | mg |
| Further inactive carriers | | | | |

2b. Formulation of Urolithin A with benefits for brain health

| | | |
|---|---|---|
| Component | Weight range | Weight |

| | | | | |
|---|---|---|---|---|
| Urolithin A | 250-2500 | mg | 500 | mg |
| Phosphatidylserine | 100-200 | mg | 125 | mg |
| Ergothioneine | 5-25 | mg | 25 | mg |
| Selenium | 30-100 | μg | 50 | μg |
| Further inactive carriers | | | | |

2c. Further formulation of Urolithin
A with benefits for brain health

| Component | Weight range | Weight |
|---|---|---|
| Urolithin A | 250-2500 mg | 500 mg |
| Phosphatidylserine | 100-200 mg | 125 mg |
| Further inactive carriers | | |

2d. Further formulation of Urolithin
A with benefits for brain health

| Component | Weight range | Weight |
|---|---|---|
| Urolithin A | 250-2500 mg | 500 mg |
| Ergothioneine | 5-25 mg | 25 mg |
| Further inactive carriers | | |

2e. Further formulation of Urolithin
A with benefits for brain health

| | | |
|---|---|---|
| Component | Weight range | Weight |

| | | | | |
|---|---|---|---|---|
| Urolithin A | 250-2500 | mg | 500 | mg |
| Selenium | 30-100 | μg | 50 | μg |
| Further inactive carriers | | | | |

2f. Formulation of Urolithin A with benefits for brain health

| | | |
|---|---|---|
| Component | Weight range | Weight |

| | | | |
|---|---|---|---|
| Urolithin A | 250-2500 | mg | 500 mg |
| Phosphatidylserine | 100-200 | mg | 125 mg |
| Ergothioneine | 5-25 | mg | 25 mg |
| Further inactive carriers | | | |

2g. Formulation of Urolithin A with benefits for brain health

| | | |
|---|---|---|
| Component | Weight range | Weight |

| | | | | |
|---|---|---|---|---|
| Urolithin A | 250-2500 | mg | 500 | mg |
| Phosphatidylserine | 100-200 | mg | 125 | mg |
| Selenium | 30-100 | μg | 50 | μg |
| Further inactive carriers | | | | |

2h. Formulation of Urolithin A with benefits for brain health

| | | |
|---|---|---|
| Component | Weight range | Weight |

| | | | | |
|---|---|---|---|---|
| Urolithin A | 250-2500 | mg | 500 | mg |
| Ergothioneine | 5-25 | mg | 25 | mg |
| Selenium | 30-100 | μg | 50 | μg |
| Further inactive carriers | | | | |

2i. Formulation of Urolithin A with benefits for brain health

| | | |
|---|---|---|
| Component | Weight range | Weight |

| | | |
|---|---|---|
| Urolithin A | 500 | mg |
| Phosphatidlyserine | 100-200 | mg |
| Ergothioneine | 5-25 | mg |
| Selenium | 30-50 | μg |
| Further inactive carriers | | |

2j. Formulation of Urolithin A with benefits for brain health

| | | |
|---|---|---|
| Component | Weight range | Weight |

| | | |
|---|---|---|
| Urolithin A | 500 | mg |
| Phosphatidlyserine | 100-200 | mg |
| Ergothioneine | 5-25 | mg |
| Selenium | 30-50 | μg |
| Vitamin B6 | 50-100 | mg |
| Vitamin B9 | 400-800 | μg |
| Vitamin B12 | 200-500 | μg |
| Further inactive carriers | | |

The formulations according to Examples 2a to 2j above may be provided as a soft gel (for example in a capsule having a shell), as a powder, or as a tablet.

### Example 3: Representative Powder or Gel Compositions with Benefits for Immune Health

For the avoidance of doubt representative compositions below may include other excipients to aid formulation or other active components.

3a. Formulation of Urolithin A with benefits for immune health

| | | |
|---|---|---|
| Component | Weight range | Weight |

| | | | | |
|---|---|---|---|---|
| Urolithin A | 250-2500 | mg | 500 | mg |
| Elderberry | 50-400 | mg | 200 | mg |
| Zinc | 5-20 | mg | 10 | mg |
| Selenium | 30-100 | μg | 50 | μg |
| Quercetin | 100-400 | mg | 250 | mg |
| Further inactive carriers | | | | |

3b. Formulation of Urolithin A with benefits for immune health

| Component | Weight range | Weight |
|---|---|---|
| Urolithin A | 250-2500 mg | 500 mg |
| Elderberry | 50-400 mg | 200 mg |
| Further inactive carriers | | |

3c. Formulation of Urolithin A with benefits for immune health

| Component | Weight range | Weight |
|---|---|---|
| Urolithin A | 250-2500 mg | 500 mg |
| Zinc | 5-20 mg | 10 mg |
| Further inactive carriers | | |

3b. Formulation of Urolithin A with benefits for immune health

| | | |
|---|---|---|
| Component | Weight range | Weight |

| | | | | |
|---|---|---|---|---|
| Urolithin A | 250-2500 | mg | 500 | mg |
| Selenium | 30-100 | μg | 50 | μg |
| Further inactive carriers | | | | |

3e. Formulation of Urolithin A with benefits for immune health

| Component | Weight range | Weight |
|---|---|---|
| Urolithin A | 250-2500 mg | 500 mg |
| Quercetin | 100-400 mg | 250 mg |
| Further inactive carriers | | |

3f. Formulation of Urolithin A with benefits for immune health

| | | |
|---|---|---|
| Component | Weight range | Weight |

| | | | |
|---|---|---|---|
| Urolithin A | 250-2500 | mg | 500 mg |
| Elderberry | 50-400 | mg | 200 mg |
| Zinc | 5-20 | mg | 10 mg |
| Optional Quercetin | 100-400 | mg | 250 mg |
| Further inactive carriers | | | |

3g. Formulation of Urolithin A with benefits for immune health

| | | |
|---|---|---|
| Component | Weight range | Weight |

| | | | | |
|---|---|---|---|---|
| Urolithin A | 250-2500 | mg | 500 | mg |
| Zinc | 5-20 | mg | 10 | mg |
| Selenium | 30-100 | μg | 50 | μg |
| Optional Quercetin | 100-400 | mg | 250 | mg |
| Further inactive carriers | | | | |

3h. Formulation of Urolithin A with benefits for immune health

| | | |
|---|---|---|
| Component | Weight range | Weight |

| | | | | |
|---|---|---|---|---|
| Urolithin A | 250-2500 | mg | 500 | mg |
| Elderberry | 50-400 | mg | 200 | mg |
| Selenium | 30-100 | μg | 50 | μg |
| Optional Quercetin | 100-400 | mg | 250 | mg |
| Further inactive carriers | | | | |

3i. Formulation of Urolithin A with benefits for immune health

| Component | Weight range | Weight |
|---|---|---|
| Urolithin A | 250-2500 mg | 500 mg |
| Quercetin | 100-400 mg | 250 mg |
| Further inactive carriers | | |

3j. Formulation of Urolithin A with benefits for immune health

| | | |
|---|---|---|
| Component | Weight range | Weight |

| | | | |
|---|---|---|---|
| Urolithin A | 250-2500 | mg | 500 mg |
| Quercetin | 100-400 | mg | 250 mg |
| Elderberry | 200 | mg | 200 mg |
| Further inactive carriers | | | |

3k. Formulation of Urolithin A with benefits for immune health

| | | |
|---|---|---|
| Component | Weight range | Weight |

| | | | |
|---|---|---|---|
| Urolithin A | 250-2500 | mg | 500 mg |
| Quercetin | 100-400 | mg | 250 mg |
| Elderberry | 200 | mg | 200 mg |
| Zinc | 5-10 | mg | |
| Selenium | 30-50 | μg | |
| Further inactive carriers | | | |

The formulations according to Examples 3a to 3k above may be provided as a soft gel (for example in a capsule having a shell), as a powder, or as a tablet.

### Example 4. Synergistic Anti-Inflammatory Effects of Urolithin a and Zinc in Human Fibroblasts (nHDF)

Chronic inflammation, also known as inflamm-aging, is a key driver of immune aging and is closely linked to the onset and progression of immune-related disorders, including autoimmune diseases, chronic infections, and impaired vaccine responses. Among the immune cytokines, dysregulated IL-6 signalling is a key contributor of immune health. There is strong evidence that IL-6 serum concentration increases with age (Wei et al (1992) Life Sci 51(25), 1953-6). The causal link between increased IL-6 levels and immune dysfunctions is corroborated by experiments showing that IL-6 antagonist receptor are very potent in reducing autoimmune diseases (Tanaka et al (2014) Cold Spring Harb Perspect Biol 6(10): a016295). In general, IL-6 contributes to immune dysfunction, promoting chronic inflammation and increasing susceptibility to disease (Hirano (2021) Int Immunol 33(3), 127-148).

To evaluate the anti-inflammatory potential of compounds relevant to immune health, we employed human dermal fibroblasts as a model system. Fibroblasts can detect and respond to inflammatory stimuli, secrete cytokines, and modulate immune cell recruitment and activation, positioning them as non-classical components of the innate immune system (Davidson et al (2021) Nat Rev Immunol 21(11), 704-717). In particular, fibroblasts are closely associated with immune cells in many tissues, where they contribute to immune surveillance, cytokine signalling, and tissue homeostasis. This close interaction makes fibroblasts a valuable model for assessing immune-modulatory effects in a tissue-agnostic manner, offering a more universal approach to evaluating immune health than models focused on a single immune cell type (Davidson et al (2021) Nat Rev Immunol 21(11), 704-717).

In this example, we present data showing that the combination of Urolithin A and zinc synergistically reduces IL-6 secretion in human dermal fibroblasts exposed to a pro-inflammatory cytokine cocktail (IL-1, TNF-α, and IFN-γ). These findings highlight the potential of this combination to modulate inflammatory pathways and support immune health.

A dose of 0.025 ng/ml of Cytokines mix was applied to induce inflammation, measured by quantifying the levels of the pro-inflammatory cytokine interleukin 6 (IL-6) in the cell culture supernatant. For the experiment, cells were first pre-treated with either i) urolithin A 2.5 uM, ii) Zinc at 100 uM, iii) a combination of urolithin A 2.5 uM and Zinc 100 uM, iv) or DMSO as control for 24 h. Then cells were co-treated with both CTX and either urolithin A or Zinc alone or in combination, as above. After 24 h, IL-6 was measured in the cell supernatant to assess the ability of Urolithin A and Zinc to reduce Cytokines-induced inflammation.

Results indicate that CTX drastically increases IL-6 levels. Both urolithin A and Zinc treatment reduce CTX-mediated increase of IL-6 by 25% and 29.6% respectively (Table 5). The combination of both Urolithin A and Zinc resulted in a further synergistic decrease of IL-6 levels by 43.7%, compared to the treatment with the compounds alone (Table 5 and Table 6 below).

TABLE 5

| | | | | Combo |
|---|---|---|---|---|
| | CTX 0.025 | | | Zinc 100 uM/ |
| | ng/ml | UA 2.5 uM | Zinc100 uM | 2.5 uM UA |
| Relative | 0.00% | −25.05% | −29.57% | −43.69% |
| Change | | | | |
| (%) | | | | |
| Compared | | | | |
| to CTX | | | | |

Table 6 indicates the change in Interleukin-6 (IL-6) concentration in the cell supernatant expressed as % change for the indicated comparisons. Corresponding statistical significance is provided as adjusted p.value, calculated by One-Way ANOVA.

TABLE 6

| | % reduction | Adjusted P |
|---|---|---|
| Ordinary One-Way ANOVA | IL-6 | Value |
| CTX vs. CTX + UA 2.5 uM | −25% | <0.023 |
| CTX vs. CTX + Zinc 100 uM | −29.6% | <0.0077 |
| CTX vs. CTX + UA 2.5 uM + Zinc 100 uM | −43.7% | <0.0002 |

### Methods

Normal Human dermal fibroblasts (nHDF) purchased from Promocell (ref. C-12302) were seeded at 6000 cells per well in a 96 well plate (Greiner) using a DMEM Glutamax 1 g D-glucose (Gibco, ref 21885108) supplemented with FBS 10% (PanBiotech, ref P30-3033), 1% PenStrep (Biowest) and 1% Hepes (Biowest). All media were sterilized with a 0.45 μm filter unit (VWR). The day after the seeded cells were treated for 24 h with 1) Urolithin A (UA) resuspended in DMSO, Zinc gluconate (MedChemExpress Ref. HY-W145499) or a combination of the two at the indicated doses.Cytokine mix (“cytomix”) was added in addition of the previous treatment for another 24 h, cytokines were used equally for a total and final concentration of 0.025 ng/ml (mIFN-g (roche ref.11276905001), TNF-α (Peprotech ref. 300-01A) and IL-b (Proteintech ref. 200-011B)). The medium was collected and an ELISA for Interleukin-6 were performed following the instruction provided by the supplier (Proteintech ref. KE10007). Finally, the absorbance at a wavelength of 450 nm was measured using a microplate plate reader (Fluostar Optima, BMG Labtech). IL-6 data were finally normalized over cell number, calculated with a cell viability assay (Promega, ref. G7571).

### Example 5: A Randomized, Double-Blind, Placebo-Controlled Study Assessing the Impact of Health and Wellness Products on Cognitive Function and Related Health Outcomes

### SUMMARY

This is a randomized, double-blind, placebo-controlled study conducted with adult participants, residing in the United States. Eligible participants will (1) endorse a desire for better concentration, (2) have the opportunity for meaningful improvement (at least 30%) in their primary health outcome, and (3) express acceptance in taking a product and not knowing its formulation until the end of the study.

Participants that report a known cardiac dysfunction, liver or kidney disease may be excluded. Participants that report a known contraindication or with well-established, significant safety concerns due to illness will be excluded. Heavy drinkers and those who report they are pregnant, trying to become pregnant, or breastfeeding will be excluded. Participants that report taking medications with a known contraindication or with well-established, significant safety concerns will be excluded.

Self-reported data are collected electronically from eligible participants for up to 13 weeks. Participant reports of health indicators will be collected at baseline, throughout the active period of study product use, and in a final survey. All study assessments will be electronic; there are no in-person visits or assessments for this real-world evidence study.

### Objectives & Outcomes

Outcomes are assessed over the study period from baseline to the end of participant engagement.
Between-group differences in effect on each outcome will be assessed between each active product group relative to the placebo control. Between group comparisons of effect may include:
   Difference in PRO score
   Odds of experiencing a (minimally) clinically important difference (CID; MCID)
Within-group changes will be assessed for each product group.

Primary (powered):
Cognitive function as assessed by PROMIS™ Cognitive Function 8A.
This questionnaire measures:
   mental acuity,
   concentration,
   attention
   verbal fluency, and
   cognitive health.

Secondary (powered):
Fatigue assessed by PROMIS™ Fatigue 4A
The questionnaire measures:
   Reduce feelings of exhaustion;
   Energy;
   Vitality; and
   Feeling motivated.
Sleep-related impairment assessed by PROMIS™ Sleep-Related Impairment 4A This questionnaire measures:
   Restful sleep;
   Sleep onset pattern;
   Feeling well rested; and
   Support for healthy sleep.
Cognitive abilities as assessed by PROMIS™ Cognitive Abilities 8A This questionnaire measures:
   Support for concentration;
   Support for attention; and
   Support for short term memory.

### Exploratory:

Identify covariates that are associated with changes in health-related outcomes from enrollment to the end of the study period
When applicable; optional:
   Evaluation of application-based cognitive function tests and their correlative value to health outcomes
   Evaluation of a direct-to-consumer biospecimen assay and its correlative value to health outcomes

### Adverse Events (AEs):

AEs (side effect/unanticipated problem) profile to include number, type, severity, causality, and outcome for each study arm and for the overall study

### Stratification and Randomization Schema:

Participants will be stratified based on their assigned sex at birth and health outcome score during enrollment, then randomized to one of the study arms. Each participant will have an equal chance of being assigned to any study arm.

### Number of Sites

### One

### Subject Participation

7, 9, or 13 weeks

### Estimated Time to Complete Enrollment

6, 8, or 12 weeks

### Test Product:

Composition comprising:
(a) Urolithin A;
(b) Phosphatidylserine;
(c) Ergothioneine; and
(d) Selenium, in the form SelenoExcel™
   (mixture of selenomethionine, selenocysteine, and methyl-selenocysteine)

### Study Design

This is a parallel group, randomized, double-blind, placebo-controlled study assessing the safety and effects of health and wellness products on self-reported poor attention/concentration (brain fog) and health in adults that reside in the USA. It is a direct-to-consumer, virtual study with no in-person visits; participants will take their assigned study product (active or placebo) for up to 12 weeks and answer electronic surveys delivered to them through a text message (SMS) link or through email. Participants will be informed of their study arm assignment at the end of the study.

Participants that complete baseline will be stratified based on their assigned sex at birth and their health outcome score during enrollment to ensure equal distribution of participants among the study arms. When applicable, participation in an optional biospecimen study will be an additional stratification parameter. The specific formulations of the study products are not a factor in stratification.

Following stratification, participants will be randomized to one of the study arms (placebo or active study product) sequentially, taking all stratification parameters into account to ensure equal distribution to each study product group. The specific formulations of the study products are not a factor in randomization.

Following randomization, participants will receive a supply of study product to their home along with a study insert detailing the instructions for use and information specific to this study. For the study duration, participants will complete electronic surveys using a mobile device or computer. Links to the surveys will be sent via text message or email.
Participants will complete an initial survey (less than 2 minutes to complete) regarding their impression of the product and effects noticed after the first serving.
Weekly, participants will complete longer surveys (15 minutes or less to complete) specific to the health outcomes in this protocol and based on endorsement at enrollment, in addition to their study product usage and effects.
Participants will complete a final questionnaire (15 minutes or less to complete) about the overall impact of their study product usage during the study, an opportunity to receive communication from the collaborating funding sponsor and from Radicle Science, and provide feedback with respect to their impressions of the study overall and motivations to join.

Upon completion of the study, participants will be unblinded and provided a study participant report summarizing the data they logged during the study. The study participant report may also contain anonymous aggregate descriptive statistics of these metrics. The report will not contain any medical advice or suggestions, but merely descriptive statistics.
After receiving their participant health report, participants will have the opportunity to provide feedback (less than 2 minutes to complete) on the report

The duration of study participation will be up to 13 weeks after completing the enrollment process.

### Assessment

### Baseline

Participants must complete their baseline assessments to successfully enroll in the study, receive a study arm assignment, and to trigger study product shipment. Baseline assessments are self-reported and include:
PROMIS™ Cognitive Function 8A
PROMIS™ Fatigue 4A
PROMIS™ Sleep-Related Impairment 4A
PROMIS™ Cognitive Abilities 8A
When applicable:
   Inclusion of up to 10 custom questions regarding participant health, experiences, and/or study product
   Optional:
      For participants that consent to optional biospecimen assays, the biospecimens may be collected during baseline (before Day 1).
      Application-based cognitive function tests at the end of the baseline survey

### Active Study Product Usage

Initial assessment:
Study Product Impression
First Serving and Effects

### Weekly Assessments

Weekly assessments are self-reported and include:
Weekly PRO Surveys
PROMIS™ Cognitive Function 8A
PROMIS™ Fatigue 4A
PROMIS™ Sleep-Related Impairment 4A
PROMIS™ Cognitive Abilities 8A
Weekly Study Satisfaction Survey
Weekly Study Product Survey
Side Effects
When applicable:
   Inclusion of up to 10 custom questions regarding participant health, experiences, and/or study product
   Optional:
      For participants that consent to optional biospecimen assays, the biospecimens may be collected during the final week of study product usage (+/−1 week).
      Application-based cognitive function tests at the end of the weekly health survey

### Last Weekly Product Usage Survey Only

The following are asked in the last weekly product usage survey or in the final survey (after the study product usage period).
PGIC: Benefits noticed over study period (may be asked in final weekly assessment during product usage)

### Study Completion

The final questionnaire will assess the study product impact over the duration of study product usage. It is self-reported and may include:
Use of caffeine, alcohol and/or cannabinoid products
Participant, health, and/or study product related questions of interest
Impression of the study product and willingness to purchase and/or recommend
Purchasing behavior with respect to health and wellness products
Desire to receive information from the collaborating brand
The study enrollment process
The surveys
Motivations to participate
Customer support
Likelihood to recommend or participate in a Radicle Study
Open feedback regarding the study product
When applicable:
   Inclusion of up to 10 custom questions regarding participant health, experiences, and/or study product
   Optional: Application-based cognitive function tests at the end of the final survey

### Example 6. Example Compositions

Compositions of the invention include compositions comprising ingredients listed in Tables 1-5. Such example compositions of the invention are listed in Tables 7-11.

TABLE 7
Brain product 1

| | | In product | | (mg/kg) | | | | | | | | |
|---|---|---|---|---|---|---|---|---|---|---|---|---|
| | | | Optimal | | | Optimal | | | Optimal | | | Optimal |
| | | | Weight | Human dose | | Human | Dog dose | | Dog | Cat Dose | | Cat |
| Ingredient | Weight range (mg) | | (mg) | range* | | Dose | range | | Dose | range | | Dose |
| Urolithin A | 250 | 2500 | 500 | 4.2 | 41.7 | 8.3 | 7.9 | 79.2 | 15.8 | 13.3 | 133.3 | 26.7 |
| Phospatidylserine | 100 | 200 | 125 | 1.7 | 3.3 | 2.1 | 3.2 | 6.3 | 4.0 | 5.3 | 10.7 | 6.7 |
| Ergotheonine | 5 | 25 | 25 | 0.1 | 0.4 | 0.4 | 0.2 | 0.8 | 0.8 | 0.3 | 1.3 | 1.3 |
| Selenium | 30 | 100 | 50 | 0.5 | 1.7 | 0.8 | 1.0 | 3.2 | 1.6 | 1.6 | 5.3 | 2.7 |
| L-methylfolate | 5 | 15 | 7.5 | 0.1 | 0.3 | 0.1 | 0.2 | 0.5 | 0.2 | 0.3 | 0.8 | 0.4 |

TABLE 8
Brain Product 2

| | | In product | | (mg/kg) | | | | | | | | |
|---|---|---|---|---|---|---|---|---|---|---|---|---|
| | | | Optimal | | | Optimal | | | Optimal | | | Optimal |
| | | | Weight | Human dose | | Human | Dog dose | | Dog | Cat Dose | | Cat |
| Ingredient | Weight range (mg) | | (mg) | range* | | Dose | range | | Dose | range | | Dose |
| Urolithin A | 250 | 2500 | 500 | 4.2 | 41.7 | 8.3 | 7.9 | 79.2 | 15.8 | 13.3 | 133.3 | 26.7 |
| Phospatidylserine | 100 | 200 | 125 | 1.7 | 3.3 | 2.1 | 3.2 | 6.3 | 4.0 | 5.3 | 10.7 | 6.7 |
| Ergotheonine | 5 | 25 | 25 | 0.1 | 0.4 | 0.4 | 0.2 | 0.8 | 0.8 | 0.3 | 1.3 | 1.3 |
| Selenium | 30 | 100 | 50 | 0.5 | 1.7 | 0.8 | 1.0 | 3.2 | 1.6 | 1.6 | 5.3 | 2.7 |
| Vitamin B6 | 20 | 100 | | 0.3 | 1.7 | | 0.6 | 3.2 | | 1.1 | 5.3 | |
| Vitamin B9 | 400 | 800 | | 6.7 | 13.3 | | 12.7 | 25.3 | | 21.3 | 42.7 | |
| Vitamin B12 | 200 | 500 | | 3.3 | 8.3 | | 6.3 | 15.8 | | 10.7 | 26.7 | |

TABLE 9
Immune product

| | | In product | | (mg/kg) | | | | | | | | |
|---|---|---|---|---|---|---|---|---|---|---|---|---|
| | | | Optimal | | | Optimal | | | Optimal | | | Optimal |
| | | | Weight | Human dose | | Human | Dog dose | | Dog | Cat Dose | | Cat |
| Ingredient | Weight range (mg) | | (mg) | range* | | Dose | range | | Dose | range | | Dose |
| Urolithin A | 250 | 2500 | 500 | 4.2 | 41.7 | 8.3 | 7.9 | 79.2 | 15.8 | 13.3 | 133.3 | 26.7 |
| Elderberry | 50 | 400 | 200 | 0.8 | 6.7 | 3.3 | 1.6 | 12.7 | 6.3 | 2.7 | 21.3 | 10.7 |
| Zinc | 5 | 20 | 10 | 0.1 | 0.3 | 0.2 | 0.2 | 0.6 | 0.3 | 0.3 | 1.1 | 0.5 |
| Selenium | 30 | 100 | 50 | 0.5 | 1.7 | 0.8 | 1.0 | 3.2 | 1.6 | 1.6 | 5.3 | 2.7 |
| Quercitin | 100 | 400 | 250 | 1.7 | 6.7 | 4.2 | 3.2 | 12.7 | 7.9 | 5.3 | 21.3 | 13.3 |

TABLE 10
Stress/Mood Product

| | | In product | | (mg/kg) | | | | | | | | |
|---|---|---|---|---|---|---|---|---|---|---|---|---|
| | | | Optimal | | | Optimal | | | Optimal | | | Optimal |
| | | | Weight | Human dose | | Human | Dog dose | | Dog | Cat Dose | | Cat |
| Ingredient | Weight range (mg) | | (mg) | range* | | Dose | range | | Dose | range | | Dose |
| Urolithin A | 250 | 2500 | 500 | 4.2 | 41.7 | 8.3 | 7.9 | 79.2 | 15.8 | 13.3 | 133.3 | 26.7 |
| Rhodiola Rosea | | | 240 | | | 4.0 | | | 7.6 | | | 12.8 |
| Lutein | | | 10 | | | 0.2 | | | 0.3 | | | 0.5 |
| L-theanine | 100 | 900 | 100 | 1.7 | 15.0 | 1.7 | 3.2 | 28.5 | 3.2 | 5.3 | 48.0 | 5.3 |
| Ashwaghanda | 250 | 600 | | 4.2 | 10.0 | 0.0 | 7.9 | 19.0 | 0.0 | 13.3 | 32.0 | 0.0 |

TABLE 11
Sleep Product

| | | In product | | (mg/kg) | | | | | | | | |
|---|---|---|---|---|---|---|---|---|---|---|---|---|
| | | | Optimal | | | Optimal | | | Optimal | | | Optimal |
| | | | Weight | Human dose | | Human | Dog dose | | Dog | Cat Dose | | Cat |
| Ingredient | Weight range (mg) | | (mg) | range* | | Dose | range | | Dose | range | | Dose |
| Urolithin A | 250 | 2500 | 500 | 4.2 | 41.7 | 8.3 | 7.9 | 79.2 | 15.8 | 13.3 | 133.3 | 26.7 |
| Boswellia serrata | | | 54 | | | 0.9 | | | 1.7 | | | 2.9 |
| Terminalia chebula | | | 260 | | | 4.3 | | | 8.2 | | | 13.9 |
| L-theanine | 100 | 900 | 900 | 1.7 | 15.0 | 15.0 | 3.2 | 28.5 | 28.5 | 5.3 | 48.0 | 48.0 |
| CBD | 100 | 900 | 300 | 1.7 | 15.0 | 5.0 | 3.2 | 28.5 | 9.5 | 5.3 | 48.0 | 16.0 |
| Melatonin | 0.5 | 10 | 5 | 0.0 | 0.2 | 0.1 | 0.0 | 0.3 | 0.2 | 0.0 | 0.5 | 0.3 |

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A composition comprising:
(a) a compound of formula (I), or a salt, prodrug, or pharmaceutically acceptable salt thereof; 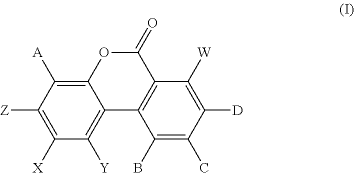
wherein:
A, B, C, D, W, X, Y and Z are each independently selected from H and OH; and
(b) one or more agents for improving brain health,
wherein the agent for improving brain health may be selected from:
(i) brain protective agents;
(ii) agents that enhance neurotransmission;
(iii) agents that promote neurogenesis (for example selenium); and/or
(iv) brain essential nutrients.

2. The composition as claimed in claim 1, comprising a brain protective agent.

3. The composition as claimed in claim 2, comprising an agent that enhances neurotransmission.

4. The composition as claimed in claim 3, comprising an agent that promotes neurogenesis.

5. The composition as claimed in claim 2, wherein the brain protective agent is a complex comprising phosphatidylcholine and phosphatidylserine.

6. The composition as claimed in claim 1, comprising
(a) a compound of formula (I), or a salt, prodrug, or pharmaceutically acceptable salt thereof;
(b) phosphatidylserine;
(c) ergothioneine; and
(d) selenium.

7. The composition as claimed in claim 6, comprising:
(a) about 100 mg to about 1000 mg urolithin A;
(b) about 50 mg to about 500 mg phosphatlidylserine;
(c) about 1 mg to about 100 mg ergothioneine; and
(d) about 1 μg to about 250 μg selenium.

8. The composition as claimed in claim 1, further comprising a brain-essential nutrient selected from Vitamin B6, Vitamin B9, Vitamin B12, magnesium salts, folic acid and/or L-methylfolate.

9. (canceled)

10. The composition as claimed in claim 9, further comprising medium chain triglycerides and/or ketones, for example, β-hydroxybutyrate (BHB) and esters of β-hydroxybutyrate.

11. A method for improving, protecting, and/or maintaining brain function and cognition, for improving, protecting, and/or maintaining cognitive function, or for enhancing mental alertness, comprising administering to a subject in need thereof an effective amount of a composition of claim 1.

12. (canceled)

13. The method as claimed in claim 11, wherein the cognitive function is selected from the group consisting of perception, memory, attention, speech comprehension, speech generation, reading comprehension, creation of imagery, learning, and reasoning.

14. (canceled)

15. A method for increasing psychomotor speed and/or reaction time, comprising administering to a subject in need thereof an effective amount of a composition of claim 1.

16. A method for enhancing/improving reasoning or focus, comprising administering to a subject in need thereof an effective amount of a composition of claim 1.

17. The method of claim 16, wherein the enhancing/improving focus comprises enhancing/improving concentration, complex attention and/or sustained attention.

18. A composition comprising:
(a) a compound of formula (I), or a salt, prodrug, or pharmaceutically acceptable salt thereof; 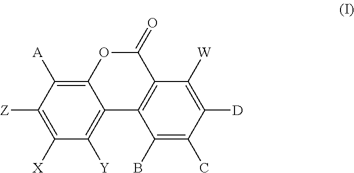
wherein:
A, B, C, D, W, X, Y and Z are each independently selected from H and OH; and
(b) one or more agents for improving immune health, wherein the one or more agents for improving immune health comprise:
(i) quercetin
(ii) Elderberry or an extract thereof;
(iii) a selenium salt; and/or
(iv) a zinc salt.

19. A composition as claimed in claim 18, further comprising one or more of the following:
(v) ginseng;
(vi) echinacea;
(vii) immuse;
(viii) bromelain;
(ix) lactoferrin;
(x) colostrum;
(xi) β-glucan;
(xii) lysine; and
(xiii) arginine

20. A method for maintaining or enhancing immune function, comprising administering to a subject in need thereof an effective amount of a composition of claim 18.

21. The method of claim 20, wherein the maintaining or enhancing of immune function comprises one or more of the following:
(a) reducing the risk of/protecting against respiratory tract infections (for example, colds, flu or pneumonia);
(b) increasing the rate of recovery from respiratory tract infections (for example, colds, flu or pneumonia);
(c) improving vaccination response;
(d) enhancing an immune response against infection;
(e) treating or for preventing immunosenescence;
(f) improving the mitochondrial function in immune cells;
(g) reducing or slowing inflammaging;
(h) increasing CD8 positive T-cells, for example, naïve CD8-positive T-cells
(i) slowing, reversing, managing immune aging and/or aging of immune cells; and/or
(j) improving metabolic function of immune cells.

22. The composition of claim 1, wherein the compound of formula (I) is urolithin A, urolithin B, urolithin C or urolithin D.

23. The composition of claim 22, wherein the compound of formula (I) is urolithin A.
